(19) United States
(12) Patent Application Publication
(10) Pub. No.: US 2026/0355269 A1
(43) Pub. Date: Nov. 12, 2026

(54) METHOD FOR PROMOTING JOINT HEALTH BY USING MIXTURE OF TYPE II COLLAGEN-MIMICKING AMINO ACID COMPOSITION AND PLANT EXTRACT

(71) Applicant: TCI CO., LTD., Taipei (TW)

(72) Inventors: YUNG-HSIANG LIN, Taipei (TW); YU-LING WANG, Taipei (TW)

(21) Appl. No.: 19/337,930

(22) Filed: Sep. 24, 2025

Related U.S. Application Data
(60) Provisional application No. 63/698,582, filed on Sep. 25, 2024.

(51) Int. Cl. A61K 36/185; A61K 31/198; A61K 31/405; A61K 31/4172; A61K 36/9066; A61P 19/02; A61P 19/10

(52) U.S. Cl. CPC A61K 36/185; A61K 31/198; A61K 31/405; A61K 31/4172; A61K 36/9066; A61P 19/02; A61P 19/10

(57) ABSTRACT

The present disclosure relates to a mixture of an amino acid composition and a plant extract, and to a method of using the same for promoting joint health. The mixture of the present disclosure can increase bone hardness, enhance bone mineral density, promote osteoblast differentiation, inhibit osteoclast differentiation, prevent inflammatory responses, reduce inflammatory factors, prevent chondrocyte inflammation, relieve joint pain, prevent joint wear, promote secretion of cartilage collagen, promote secretion of glucosaminoglycans, and/or improve joint mobility and relieve joint stiffness, thereby providing systemic support for bone and joint health.

### CROSS-REFERENCE TO RELATED APPLICATION

This application claims the benefit of U.S. provisional application Ser. No. 63/698,582, filed on Sep. 25, 2024. The entirety of the above-mentioned patent application is hereby incorporated by reference herein and made a part of the specification.

### REFERENCE OF AN ELECTRONIC SEQENCE LISTING

The contents of the electronic sequence listing (P246462USI.xml; Size: 13,559 bytes; and Date of Creation: Sep. 22, 2025) is herein incorporated by reference in its entirety.

### BACKGROUND

### Technical Field

The present disclosure relates to a mixture of an amino acid composition and a plant extract and application thereof, and also relates to a joint health care mixture including the amino acid composition and the plant extract.

### Related Art

Collagen is an important structural protein in connective tissues of a human body, and is widely found in tissues such as skin, cartilage, bones, and joints. Among which, type II collagen plays a key role in maintaining the structure and function of cartilage tissue, and can support the elasticity and stability of the joints. As people aged, the natural synthesis of collagen in the human body gradually decreases, the reduction of collagen in bone tissue may weaken the bone matrix, and increase the risk of bone loss and fractures. Therefore, supplementing or promoting collagen production has become an important means to delay skin aging and maintain bone health.

At present, most collagen supplements are derived from animal tissues (such as cows, pigs, fish skin, or fish scales) and are hydrolyzed into small peptides to improve bioavailability. However, these products have several limitations, including potential allergic reactions, the risk of pathogen transmission, and that some consumers cannot accept animal-derived ingredients due to religious, ethical, or dietary reasons. In addition, from the perspectives of environment and sustainable development, relying heavily on animal sources also has certain constraints.

To solve the above problems, a concept of biomimetic collagen has been proposed in recent years. The biomimetic collagen is prepared by a plant-based biomimetic technology simulating the amino acid composition of human collagen, which provides a purely plant-derived, sustainable, and allergy-free collagen option.

Bone and joint health is the foundation for normal functioning of the human motion system, and the maintenance thereof depends on the balance of bone tissue metabolism, the integrity of cartilage, and the lubrication of joint cavities. However, with the increasing trend of nationwide fitness and aging of population, joint problems are no longer limited to the elderly but are gradually affecting younger and more active individuals. Young people experience decreased bone density and bone hardness in knee joints, hip joints, and spine due to prolonged desk work, lack of sunlight, and insufficient calcium intake. Fitness enthusiasts are prone to micro-damage of cartilage in knee joints, hip joints, and ankle joints due to intense exercise, and if not adequately repaired, this may lead to early onset of joint stiffness and joint pain. Elderly people are prone to bone loss due to a decrease in osteoblast activity and an increase in osteoclast activity, leading to an imbalance in bone remodeling, and meanwhile, reduced synthesis of cartilage matrix and narrowing of joint cavities may result in joint stiffness, pain, and restricted activity.

Therefore, there is an urgent need to develop a joint health care composition that can increase bone hardness, prevent inflammatory responses, relieve joint pain, and/or improve joint mobility, thereby providing systemic support for bone and joint health.

### SUMMARY

In view of the above problems and demands in the field, the present disclosure provides a type II human collagen-mimicking amino acid composition. A mixture of the amino acid composition and a berry extract (such as an elderberry extract) and/or a Curcuma L. plant extract (such as a Curcuma longa extract) can achieve a synergistic effect, so as to increase bone hardness, prevent inflammatory responses, relieve joint pain, and/or improve joint mobility.

In a first aspect, the present disclosure provides an amino acid composition. The amino acid composition includes the following amino acids or salts thereof in weight percentage:
20.0%-28.0% of glycine, 13.0%-18.0% of proline, 6.0%-10.0% of alanine, 4.0%-7.0% of aspartic acid, 4.0%-7.0% of glutamine, 3.5%-6.0% of glutamic acid, 3.5%-5.0% of arginine, 3.0%-4.5% of lysine, 3.0%-4.5% of serine, 2.5%-4.0% of leucine, 2.0%-3.2% of threonine, 1.8%-2.8% of valine, 1.5%-2.5% of methionine, 1.5%-2.5% of isoleucine, 1.0%-2.0% of phenylalanine, 0.4%-0.8% of tyrosine, 0.3%-0.6% of histidine, 0.3%-0.6% of tryptophan, and an optional additive.

In one embodiment, the additive accounts for 1.5%-6.0% relative to the total weight of the amino acid composition, and the additive includes 3.0%-5.0% of maltodextrin and/or 2.0%-4.0% of vitamin C.

In one embodiment, the amino acid composition includes the following amino acids or salts thereof in weight percentage: 24.03% of glycine, 15.98% of proline, 7.93% of alanine, 5.56% of aspartic acid, 5.44% of glutamine, 4.68% of glutamic acid, 4.26% of arginine, 3.97% of lysine, 3.96% of serine, 3.31% of leucine, 2.60% of threonine, 2.25% of valine, 2.07% of methionine, 2.01% of isoleucine, 1.48% of phenylalanine, 0.59% of tyrosine, 0.47% of histidine, 0.41% of tryptophan, 2.25% of maltodextrin, and 3.00% of vitamin C.

In one embodiment, the amino acid composition includes the following amino acids or salts thereof in weight percentage: 24.03% of glycine, 15.98% of proline, 7.93% of alanine, 5.56% of aspartic acid, 5.44% of glutamine, 4.68% of glutamic acid, 4.26% of arginine, 3.97% of lysine, 3.96% of serine, 3.31% of leucine, 2.60% of threonine, 2.25% of valine, 2.07% of methionine, 2.01% of isoleucine, 1.48% of phenylalanine, 0.59% of tyrosine, 0.47% of histidine, 0.41% of tryptophan, 3.25% of maltodextrin, and 3.00% of vitamin C.

In one embodiment, the amino acid composition includes the following amino acids or salts thereof in weight percentage: 24.03% of glycine, 15.98% of proline, 7.93% of alanine, 5.56% of aspartic acid, 5.44% of glutamine, 4.68% of glutamic acid, 4.26% of arginine, 3.97% of lysine, 3.96% of serine, 3.31% of leucine, 2.60% of threonine, 2.25% of valine, 2.07% of methionine, 2.01% of isoleucine, 1.48% of phenylalanine, 0.59% of tyrosine, 0.47% of histidine, 0.41% of tryptophan, 5.00% of maltodextrin, and 3.00% of vitamin C.

In one embodiment, each amino acid component in the amino acid composition is derived from industrial sources, renewable plant sources, or prepared from glucose by fermentation.

In a second aspect, the present disclosure provides a mixture of the amino acid composition in the first aspect and a berry extract, and a method for increasing bone hardness, and the method includes: administering an effective dose of the mixture to a subject in need thereof.

In one embodiment, the berry extract is an elderberry extract.

In one embodiment, the mixture is used for enhancing bone mineral density.

In one embodiment, the mixture is used for promoting osteoblast differentiation.

In one embodiment, the mixture is used for inhibiting osteoclast differentiation.

In a third aspect, the present disclosure provides a mixture of the amino acid composition in the first aspect and a berry extract, and a method for preventing inflammatory responses, and the method includes: administering an effective dose of the mixture to a subject in need thereof.

In one embodiment, the berry extract is an elderberry extract.

In one embodiment, the mixture is used for reducing a level of inflammatory factors. In one embodiment, the composition is used for reducing levels of TNF-α, IL-1β, and iNOS.

In one embodiment, the mixture is used for preventing chondrocyte inflammation.

In a fourth aspect, the present disclosure provides a mixture of the amino acid composition in the first aspect and a Curcuma plant extract, and a method for relieving joint pain, and the method includes: administering an effective dose of the mixture to a subject in need thereof.

In one embodiment, the Curcuma plant extract is a Curcuma longa extract.

In one embodiment, the mixture is used for preventing joint wear.

In one embodiment, the mixture is used for promoting secretion of cartilage collagen.

In one embodiment, the mixture is used for promoting secretion of glucosaminoglycans.

In a fifth aspect, the present disclosure provides a mixture of the amino acid composition in the first aspect and a Curcuma plant extract, and a method for improving joint mobility, and the method includes: administering an effective dose of the mixture to a subject in need thereof.

In one embodiment, the Curcuma plant extract is a Curcuma longa extract.

In one embodiment, the mixture is used for relieving joint stiffness.

In one embodiment, the mixture is used for promoting secretion of cartilage collagen.

In one embodiment, the mixture is used for promoting secretion of glucosaminoglycans (GAGs).

In one embodiment, the mixture includes a berry extract, and based on the total weight of the amino acid composition and the berry extract being 100%, the berry extract accounts for 2.0%-3.5% in weight percentage.

In one embodiment, the mixture includes the Curcuma plant extract, and based on the total weight of the amino acid composition and the Curcuma plant extract being 100%, the Curcuma plant extract accounts for 0.5%-3.0% in weight percentage.

In one embodiment, the mixture includes the berry extract and the Curcuma plant extract, and based on the total weight of the amino acid composition, the berry extract and the Curcuma plant extract being 100%, the berry extract accounts for 2.0%-3.5% in weight percentage, and the Curcuma plant extract accounts for 0.5%-3.0% in weight percentage.

In one embodiment, the berry extract is derived from fruits of Sambucus nigra.

In one embodiment, the Curcuma plant extract is derived from rhizomes of Curcuma longa.

In one embodiment, the composition or mixture includes bromatologically acceptable excipients.

In one embodiment, the bromatologically acceptable excipients include, but are not limited to, a carrier/diluent, such as maltodextrin, starch, lactose, glucose, fructose, mannitol, sorbitol, and dietary fiber; an adhesive, such as Arabic gum, gelatin, pectin, hydroxypropyl methyl cellulose (HPMC), and guar gum; a disintegrating agent (used for tablets/capsules), such as croscarmellose sodium, starch derivatives, and microcrystalline cellulose; a lubricant/antitackiness agent, such as magnesium stearate, silicon dioxide, and talcum powder; a coating agent (such as tablets or granules), such as hydroxypropyl methyl cellulose, polyvinyl alcohol, and shellac; a sweetening agent/flavoring agent, such as xylitol, erythritol, stevioside, a momordica grosvenori extract, and honey powder; spice/essence, such as lemon powder, orange juice powder, and vanilla powder; a coloring agent (natural source), such as beet red, chlorophyll, curcumin, and carotene; an anti-caking agent, such as tricalcium phosphate, calcium carbonate, and silicon dioxide; and a beverage matrix (such as powdered beverage, and functional beverage type), such as whey protein, vegetable protein, fruit juice powder, coconut powder, and oat powder.

In one embodiment, the bromatologically acceptable excipients are food-grade carriers, adhesives, antitackiness agents, and/or natural sweetening agents.

In one embodiment, the composition or mixture is in a form of powder, tablet, capsule, softgel, granule, oral liquid, pill, or suspension, and preferably is powder.

According to the present disclosure, the amino acid composition, and compositions including the same, have the following advantages:

The amino acid composition or mixture according to the present disclosure can increase bone hardness, prevent inflammatory responses, relieve joint pain, and improve joint mobility.

The amino acid composition or mixture according to the present disclosure can activate expression of genes related to collagen synthesis in chondrocytes, and promote secretion of collagen, chondroitin and hyaluronic acid in cartilage.

The amino acid composition or mixture according to the present disclosure can inhibit nitric oxide (NO) produced by inflammatory macrophages, reduce secretion of pro-inflammatory substances, and reduce the level of inflammatory factors, thereby relieving joint discomfort and protecting joint tissues.

The amino acid composition or mixture according to the present disclosure can inhibit cartilage degradation, significantly reduce inflammatory responses of chondrocytes, and inhibit a degeneration process of joint cartilage.

The amino acid composition or mixture according to the present disclosure can promote osteoblast differentiation, enhance bone formation, and also inhibit osteoclast differentiation and maturation, thereby preventing osteoporosis.

The amino acid composition or mixture according to the present disclosure can be used as health care products, food products, or food additives for non-therapeutic purposes, to increase bone hardness, prevent inflammatory responses, relieve joint pain, and improve joint mobility, in other words, the amino acid composition or mixture can also be used for treating and preventing osteoporosis, treating arthritis, relieving joint pain, and relieving activity limitations caused by joint problems.

### BRIEF DESCRIPTION OF THE DRAWINGS

FIG. 1 shows an effect of Sample 1 on expression levels of collagen-related genes in chondrocytes.

FIG. 2A shows an expression condition of glucosaminoglycans (including chondroitin and hyaluronic acid) in chondrocytes stained with Alcian blue.

FIG. 2B shows quantitative analysis for glucosaminoglycan expression in chondrocytes in FIG. 2A.

FIG. 3 shows comparison of nitric oxide (NO) production of different treatment groups in an LPS-induced macrophage model.

FIG. 4 shows a detection result of a qPCR experiment, namely, comparison of gene expression levels of various inflammatory factors in chondrocytes in different treatment groups.

FIG. 5A shows an experimental result of Sample 1 on osteogenic differentiation of mesenchymal stem cells, including Alizarin red S staining results for cells in a control group and an experimental group (Sample 1).

FIG. 5B shows quantitative analysis for osteoblast differentiation in FIG. 5A.

FIG. 6 shows an inhibitory effect of Sample 1 on osteoclast differentiation and maturation, including fluorescence microscopy observation results of a control group and an experimental group (Sample 1).

FIG. 7 shows a T-score of spines of subjects after taking Sample 2 for 12 consecutive weeks.

FIG. 8 shows a Z-score of spines of subjects after taking Sample 2 for 12 consecutive weeks.

FIG. 9 shows a serum TNF-α level in subjects after taking Sample 2 for 12 consecutive weeks.

FIG. 10 shows WOMAC scores of subjects after taking Sample 3 for 12 consecutive weeks.

### DETAILED DESCRIPTION

Unless otherwise defined, all technical and scientific terms used herein have the same meanings as commonly understood by those of ordinary skill in the art to which the present disclosure belongs.

Herein, the terms “comprise”, “have”, “include”, and “contain” shall be interpreted as open-ended terms (i.e., “including but not limited to”).

In the present disclosure, the term “amino acid”, unless otherwise specified, should include its salt forms, such as sodium salt, potassium salt, magnesium salt, hydrochloride, and sulfate.

The present disclosure is further described in detail below in conjunction with the accompanying drawings and examples. The following examples are provided for illustrative purposes only and are not intended to limit the scope of the present disclosure. Experimental methods in the examples that do not specify specific conditions are conducted under conventional conditions known in the relevant technical field, or according to the conditions recommended by the manufacturer.

### Example 1: Preparation of a Type II Collagen-Mimicking Amino Acid and Plant Extract Composition

24.03% of glycine, 15.98% of L-proline, 7.93% of L-alanine, 5.56% of L-aspartic acid, 5.44% of L-glutamine, 4.68% of L-glutamic acid, 4.26% of L-arginine, 3.97% of L-lysine hydrochloride, 3.96% of L-serine, 3.31% of L-leucine, 2.60% of L-threonine, 2.25% of L-valine, 2.07% of L-methionine, 2.01% of L-isoleucine, 1.48% of L-phenylalanine, 0.59% of L-tyrosine, 0.47% of L-histidine, 0.41% of L-tryptophan, 2.75% of elderberry extract (obtained by extracting fruits of Sambucus nigra, purchased from Hunan Huacheng Biotech, Inc. (Huachengbio)), 1.00% of Curcuma longa extract (obtained by extracting rhizomes of Curcuma longa, purchased from ARJUNA NATURALPRIVATE LTD), 2.25% of maltodextrin, and 3.00% of vitamin C were added, in weight percentage, into a mixer for mixing to obtain a primary mixture. The primary mixture was sieved by a sieve (with the pore size of 40 meshes) to obtain a fine and uniform intermediate mixture. The intermediate mixture was homogenized (homogenizer: BAON, G560 (2-434437)) to obtain a uniform mixed product, namely the type II collagen-mimicking amino acid and plant extract composition (Sample 1) in Example 1.

### Example 2: Promotion of Collagen Secretion by Chondrocytes

The Sample 1 obtained in Example 1 was used in a chondrocyte line experiment to assess the impact thereof on collagen gene expression.

Specifically, a mouse cartilage precursor cell strain ATDC5 (Sigma, 99072806) was inoculated into a 24-well culture plate at a density of 1×10⁵ cells/well, 10% of FBS (Gibco, M3009125RP) and 1% of antibiotic AA (Simply, CC501-0100) were added into a DMEM/F12 culture medium (Gibco, 3086139), and cultured in an incubator containing 5% of CO₂ at 37 °C for 24 h.

Then, a composition to be tested was respectively added into the culture medium, and grouped as follows:
Control group: blank culture medium
Experimental group: Sample 1 with concentration of 0.0625 mg/mL

After cells were continuously cultured for 48 h, the culture medium was removed, the cells were cleaned with 1×DPBS, 600 μL of RB Buffer (Geneaid, RB300) was added into each well to lyse the cells, and total RNA was extracted. The obtained RNA was used in subsequent qPCR detection to assess the expression level of collagen-related genes. Experimental data was subjected to statistical analysis by Student's t-test in Excel (** indicates P<0.01 relative to the control group, and *** indicates P<0.001 relative to the control group).

Primer sequences of each gene are shown in Table 1 below:

TABLE 1

| | Forward | Reverse |
|---|---|---|
| | sequence | sequence |
| Target gene | (5′-3′) | (5′-3′) |
| Collagen II | GCTGGTGAAGA | CCATCTTGACCT |
| (Col II A2) | AGGCAAACG | GGGAATCCAC |
| | AG | (SEQ ID NO: 2) |
| | (SEQ ID NO: 1) | |
| Collagen IX A2 | GGGATACAGAG | GGGGTACAGCAG |
| (Col IX A2) | CAGAGACCC | AGAGATAG |
| | (SEQ ID NO: 3) | (SEQ ID NO: 4) |
| Collagen IX A3 | TTCGACTTCT | TCCACTGGCCTG |
| (Col IX A3) | CTCCAGCCGA | ATCCATGT |
| | (SEQ ID NO: 5) | (SEQ ID NO: 6) |
| Collagen XI A2 | TTGCTGCTGT | TGCTTCTGCTGAT |
| (Col XI A2) | ACCTTTCG (SEQ | CTCACTT |
| | ID NO: 7) | (SEQ ID NO: 8) |

Collagen, as an important structural component of joint cartilage, can enhance the toughness and elasticity of cartilage, reduce friction on the cartilage surface during exercise, and relieve vibration and impact caused by exercise, thereby further improving joint function performance, significantly improving joint mobility, and preventing joint wear, thus relieving joint pain.

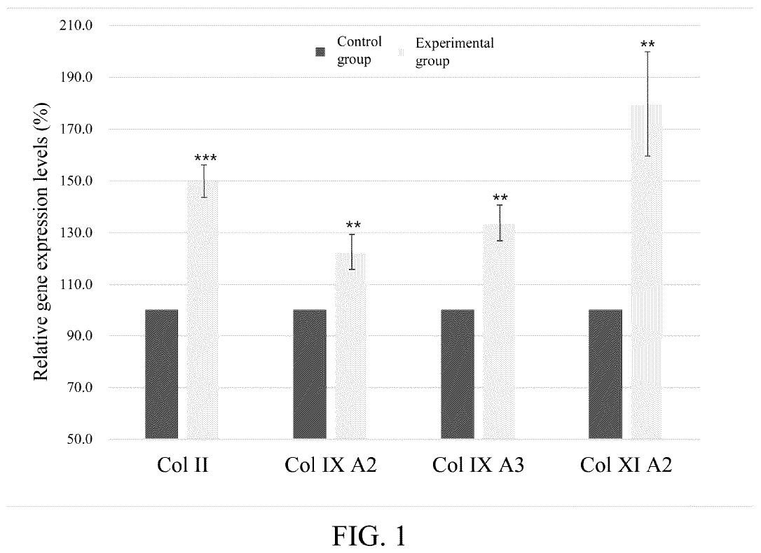

As shown in FIG. 1, experimental results showed that the Sample 1 could up-regulate the expression levels of Collagen II and other collagen-related genes (Collagen IX A2, Collagen IX A3, and Collagen XI A2). Based on the expression level of the control group (set as 100%), the relative expression level of the Collagen II gene was significantly up-regulated by 49.8%, the relative expression level of the Collagen IX A2 gene was significantly up-regulated by 22.6%, the relative expression level of the Collagen IX A3 gene was significantly up-regulated by 33.8%, and the relative expression level of the Collagen XI A2 gene was significantly up-regulated by 79.7%. The results show that the Sample 1 can promote secretion of chondrocyte collagen, and has significant effects on improving cartilage matrix synthesis, promoting joint repair, and maintaining joint health.

### Example 3: Promotion of Glucosaminoglycan Secretion by Chondrocytes

The Sample 1 obtained in Example 1 was used in a chondrocyte experiment to assess the impact thereof on secretion of glucosaminoglycans (GAGs).

Specifically, a human immortalized chondrocyte strain C28/I2 (Merck, SC043) was inoculated into a 24-well culture plate at a density of 1×10⁵ cells/well, 10% of FBS (Gibco, M3009125RP) and 1% of antibiotic AA (Simply, CC501-0100) were added into a DMEM/F12 culture medium (Gibco, 3086139), and cultured in an incubator containing 5% of CO₂ at 37 °C for 24 h.

Then, a sample to be tested was respectively added into the culture medium, and grouped as follows:
Control group: blank culture medium
Experimental group: Sample 1 with concentration of 0.0625%

Culturing was continued for 48 h, then the culture medium was removed, rinsing was carried out with 1×PBS for 3 times, then 0.5 mL of 10% paraformaldehyde was added into each well, and fixation was performed at room temperature for 30 min. After stationary liquid was removed, rinsing was carried out again with 1×PBS for 3 times. Then, 0.1 M of 1% Alcian blue solution (pH=2.5; Sigma, A5268-10G) prepared from HCl was used for staining, 0.5 mL was added into each well, and reaction was performed at room temperature overnight.

After staining, cleaning was carried out 3 times with 1×PBS, and a blue signal presented by a combination of Alcian blue and glucosaminoglycans was observed under a microscope (ZEISS Axiovert, ATCID00055). Further, 1 mL of 6 M Guanidine-HCl (Sigma, G4505-25G) was added into each well, a stain was dissolved out after acting at room temperature for 10 min, then 200 μL of cell was transferred into a 96-well plate, and the optical density at 620 nm was measured under a microplate reader (BIOTECH, Epoch1703289). Experimental data was subjected to statistical analysis by Student's t-test in Excel (* indicates P<0.05 relative to the control group).

The glucosaminoglycans are important components of joint synovial fluid and cartilage matrix, an increase in glucosaminoglycans not only helps strengthen the structure of the cartilage matrix but also enhances the lubricating function of the synovial fluid in the joint cavity, and therefore, friction can be effectively reduced during joint movement, joint smoothness can be improved, furthermore, joint mobility can be improved, and joint wear can be prevented.

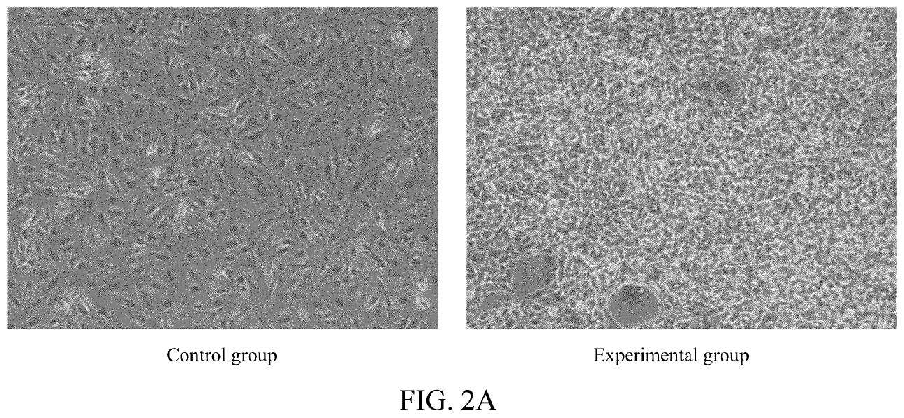

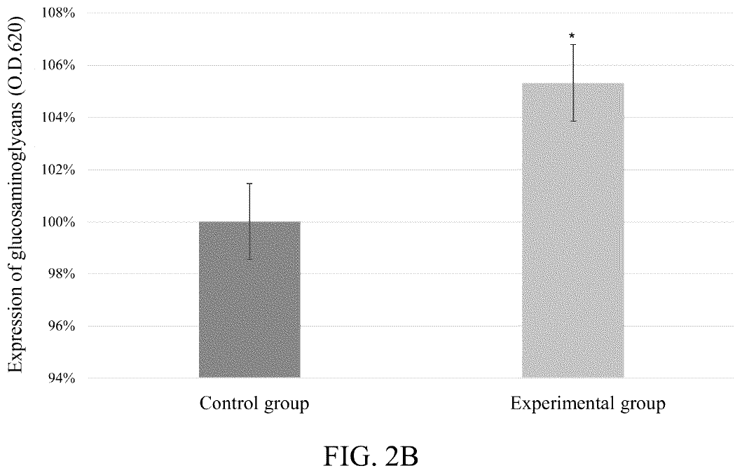

Experimental results as shown in FIG. 2A show that after the treatment group with the Sample 1 and chondrocytes were co-cultured, a GAGs staining signal (light color) was obviously enhanced under the microscope, and the light color part of the experimental group was obviously increased, which indicates that the Sample 1 could promote the chondrocytes to secrete glucosaminoglycans. As shown in FIG. 2B, according to data obtained from the microplate reader, compared with the control group, the content of glucosaminoglycans secreted by the chondrocytes was increased by 5.3%, which indicates that the Sample 1 could promote the chondrocytes to secrete glucosaminoglycans, therefore, joint wear can be effectively prevented, joint pain is relieved, and joint mobility is improved by increasing joint lubrication.

### Example 4: Inhibition of Nitric Oxide (NO) Produced by Inflammatory Macrophages

The Sample 1 obtained in Example 1 was used in a macrophage experiment to assess a regulation effect thereof on the production of inflammation-related nitric oxide (NO).

Specifically, a mouse macrophage strain RAW264.7 (ATCC, TIB-71) was selected, and 10% of FBS (Gibco, M3009125RP) and 1% of antibiotic AA (Simply, CC501-0100) were added into a DMEM culture medium (Gibco, 3124477) for culture. Cells were inoculated into a 96-well culture plate at the density of 1×10⁴ cells/well, 200 μL of culture solution was added into each well, and cultured in an incubator containing 5% of CO₂ at 37 °C for 24 h.

After culturing, the culture solution was removed and replaced with an FBS-free culture medium, and then grouping was as follows:
Control group: 500 ng/ml of lipopolysaccharide (LPS; Sigma, SI-L2880-25 MG) was added.
Experimental group: 500 ng/ml of LPS and Sample 1 with concentration of 2 mg/mL.

After culturing was continued for 24 h, 150 μL of culture supernatant was taken out from each well and transferred into a new 96-well plate, and each well was filled with 130 μL of sterile distilled water in advance. Then, a Griess agent (an agent A and an agent B were mixed according to a ratio of 1:1; Invitrogen, G7921) was prepared, 20 μL of the Griess agent was added into each well, and a light-shielding reaction was carried out for 30 min. Then, the optical density (OD) at 548 nm was measured by the microplate reader (BIOTECH, Epoch1703289). The higher the OD was, the higher the NO content concentration was. Experimental data was subjected to statistical analysis by Student's t-test in Excel (* indicates P<0.05 relative to the control group).

In an inflammatory state, macrophages could produce nitric oxide, and stimulate other immune cells to further participate in the inflammatory responses, which intensified the inflammatory process, resulting in joint damage and degeneration.

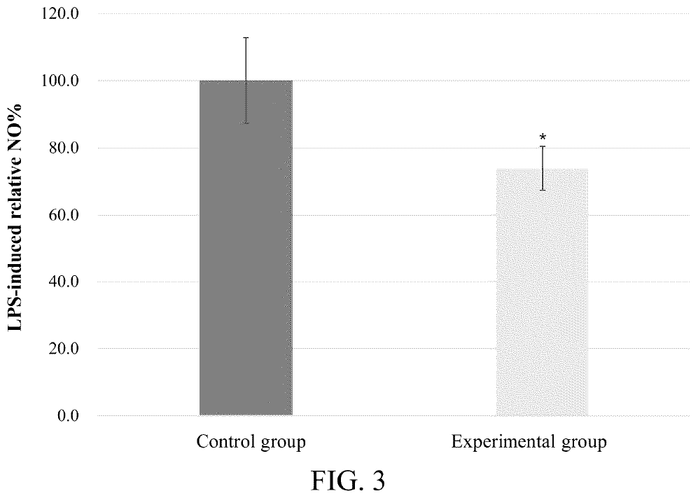

As shown in FIG. 3, experimental results showed that compared with the control group only containing LPS, the treatment group with the Sample 1 added could effectively reduce the nitric oxide produced by macrophages by 30%, which indicated that the composition could effectively inhibit macrophages from releasing nitric oxide in the inflammatory state, and therefore the inflammatory responses can be prevented.

### Example 5: Reduction of Inflammatory Responses of Chondrocytes

The Sample 1 obtained in Example 1 was used in a chondrocyte inflammation model experiment to assess a regulation effect thereof on the expression of inflammation-related factors.

Specifically, a mouse chondrocyte line ATDC5 (Sigma, 99072806) was selected, cells were inoculated into a 6-well culture plate at the density of 1×10⁵ cells/well, and cultured at the condition of 5% of CO₂ at 37 °C for 24 h. After the cells were adhered to the wall, 200 μg/mL of monosodium urate (MSU; Sigma, U2875) was added into each well to induce inflammatory responses. Then, the sample of the experimental group was added, and cultured in an incubator containing 5% of CO₂ at 37 °C for 48 h, the grouping was as follows:
Control group: MSU with concentration of 200 μg/mL.
Experimental group: MSU with concentration of 200 μg/mL and Sample 1 with concentration of 0.125 mg/mL.

After culturing, the culture solution was removed, washing was performed with 1×DPBS, and 600 μL of RB Buffer was added into each well to lyse the cells. Then, total RNA was extracted, and reverse transcription and qPCR gene quantitative analysis were carried out. Experimental data was subjected to statistical analysis by Student's t-test in Excel (*** indicates P<0.001 relative to the control group).

Detection target genes include inflammatory factors IL-1β, TNF-α and inducible nitric oxide synthase (iNOS), and primer sequences thereof are shown in Table 2 below:

TABLE 2

| Target | Forward sequence | Reverse sequence |
|---|---|---|
| gene | (5′-3′) | (5′-3′) |
| IL-1β | TTGGGCCTCA | TGTGAGGTGCTG |
| | AAGGAAAGAA | ATGTACCAGTT |
| | (SEQ ID NO: 9) | (SEQ ID NO: 10) |
| TNF-α | CCCAAGGCGC | CACCCCGAAGTT |
| | CACATCT | CAGTAGACAGA |
| | (SEQ ID NO: 11) | (SEQ ID NO: 12) |
| iNOS | CCTCAGTTCT | TGAAGCGTTT |
| | GCGCCTTTG | CGGGATCTG |
| | (SEQ ID NO: 13) | (SEQ ID NO: 14) |

When chondrocytes are damaged due to various reasons (such as aging, mechanical wear), the damaged cells may release a large amount of pro-inflammatory factors, such as IL-1β, TNF-α, and nitric oxide (NO). These signal molecules may cause inflammatory responses, and a sustained inflammatory environment may further inhibit cartilage repair and accelerate cell apoptosis, leading to more extensive joint and cartilage degeneration, ultimately resulting in restricted joint function.

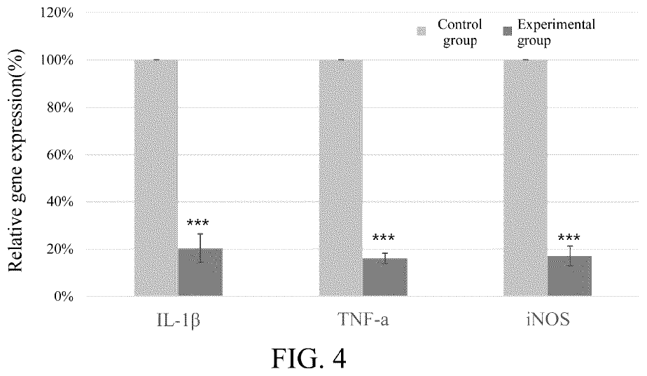

As shown in FIG. 4, experimental results showed that the Sample 1 could significantly lower the expression levels of various inflammatory factors in chondrocytes, and based on the expression level of the control group (set as 100%), the relative expression level of the IL-1β gene was significantly reduced by 80%, the relative expression level of the TNF-α gene was significantly reduced by 84%, and the relative expression level of the iNOS gene was significantly reduced by 83%. Therefore, the Sample 1 can effectively inhibit chondrocyte inflammatory responses and reduce the expression of pro-inflammatory factors, so as to play an anti-inflammatory protection role, thereby effectively delaying the process of joint cartilage degradation.

### Example 6: Promotion of Osteoblast Differentiation

The Sample 1 obtained in Example 1 was used in an osteoblast differentiation experiment to assess the role thereof in bone health.

First, human fat-derived mesenchymal stem cells AdMSC (ATCC, CRL-2749) were inoculated into a 24-well culture plate at a density of 8×10⁴ cells/well, and the culture solution volume was 500 μL/well, 5% of fetal bovine serum FBS (Gibco, M3009125RP) and 1% of antibiotic AA (Simply, CC501-0100) were added into an αMEM medium (Gibco, 12000-022), and cultured in a CO₂ culture box for 1 day. Then the culture solution was replaced with an MSCgo™ osteogenic differentiation culture medium (05-440-1B), differentiation was continued for 7 days, and the differentiation culture solution was replaced once every 2-3 days.

After 7 days, a sample to be tested was added, and grouped as follows:
Control group: blank culture medium.
Experimental group: Sample 1 with concentration of 0.5%.

After the sample was added, culturing was continued for 14 days, and the culture solution was replaced once every 2-3 days. After culturing, the culture solution was removed, the cells were cleaned twice with PBS, and then fixation was performed at 37 °C for 10 min with 4% paraformaldehyde. The fixed cells were cleaned twice with ddH₂O, and an Alizarin red S staining solution (2 g Alizarin red S/100 mL ddH₂O; Sigma, A5533) was added for staining for 1 min. After the staining solution was removed, the cells were fully washed with ddH₂O, thus obtaining a staining result. The cell calcific nodules were observed by a microscope (ZEISS Axiovert, ATCID00055).

Further, the stained sample was added into a CPC solution (350 mg CPC/10 mL ddH₂O; Sigma, C9002-25G) and acted at 37 °C for 2 h to dissolve out an Alizarin red S stain. Then, 50 μL of solution was taken out from each well and transferred into a 96-well plate, and an OD₄₀₅ value was detected by a microplate reader (BIOTECH, Epoch1703289), so as to quantitatively assess the cell calcification degree. Experimental data was subjected to statistical analysis by Student's t-test in Excel (* indicates P<0.05 relative to the control group).

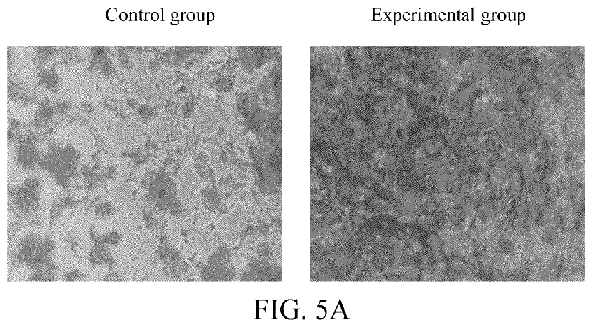

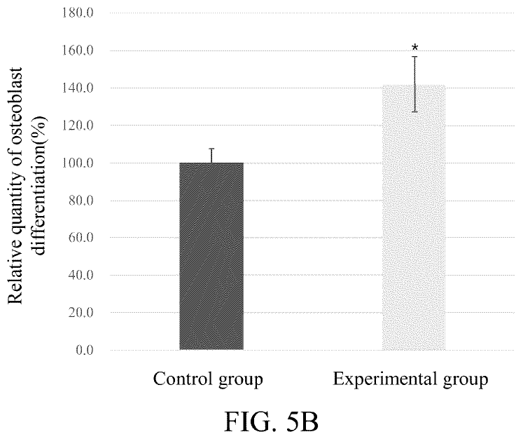

Calcium in bones was stained into red (dark color) by the Alizarin red S, as shown in FIG. 5A, the red color in the figure was displayed as dark color, and it was observed under the microscope that the red (dark color) in the experimental group was increased, which indicates that the Sample 1 could promote osteoblast differentiation. Through quantitative detection on the cell calcification degree, the capability of the Sample 1 for promoting osteoblast differentiation was indicated, the result was as shown in FIG. 5B; and according to data obtained by the microplate reader, based on the control group (set as 100%), the Sample 1 could remarkably increased the relative number of osteoblast differentiation by 41.9%. The osteoblasts are main functional cells for bone formation and are responsible for synthesis, secretion and mineralization of bone matrix; and human bone tissues are continuously reconstructed, the bone reconstruction process includes decomposition and absorption of bones and formation of new bones, and the osteoblasts are responsible for new bone formation. Through new bone formation, the bone density of an organism can be enhanced, and the bone quality is improved, so that the bone hardness is improved, and the bone health of the organism is promoted.

### Example 7: Inhibition of Differentiation and Maturation of Osteoclasts

The Sample 1 obtained in Example 1 was used in an in-vitro experiment to assess the impact on differentiation of peripheral blood mononuclear cells (PBMCs) into osteoclasts.

First, peripheral blood was collected from healthy volunteers, and 20 mL of blood was diluted with 15 mL of PBS. 15 mL of Ficoll (GE Healthcare) was added into a 50 mL centrifuge tube, 35 mL of diluted blood was slowly superposed on the Ficoll layer, and centrifuging was performed at 400 g for 40 min. A thin layer between the Ficoll layer and a plasma layer could be observed after centrifugation, and the layer was the peripheral blood mononuclear cells (PBMCs). The PBMCs were carefully transferred, and cleaning was performed twice (400 g, 15 min) with PBS.

The obtained PBMCs were inoculated at the density of 1×10⁶ cells/mL to 2×10⁶ cells/mL, and an osteoclast differentiation culture medium and the Sample 1 were added for culture. The osteoclast differentiation culture medium was prepared by adding 10% of FBS (Gibco, M3009125RP), 1×P/S, human RANKL (40 ng/mL, Peprotech, 310-01-10UG) and human M-CSF (25 ng/mL, Peprotech, 300-25-10UG) in α-MEM (Gibco, 12000-022). The cells were continuously cultured in an incubator containing 5% of CO₂ at 37 °C for 14 h, and the culture solution was replaced once every 3 days.

The grouping was as follows:
Control group: blank culture medium.
Experimental group: Sample 1 with concentration of 5 mg/mL.

After the culturing was continued for 14 days, the formation of a large amount of multinuclear osteoclasts could be observed in the control group. Then a drop of Actin Green™ 488 ReadyProbes® Reagent (Invitrogen, A11008) and a drop of Hoechst 33342 (Thermo, 62249, diluted at 1:20000) were added, and acted at 37 °C for 15 min for performing fluorescent staining on the cells, and the expression level of the osteoclasts in each group was observed by a fluorescence microscope (ZEISS Axiovert, ATCID00055).

In the bones of young individuals, the activities of osteoblasts and osteoclasts are in a dynamic balance, maintaining bone health and density. However, as people aged, the levels of cellular hormones in the body may change, leading to excessive proliferation and abnormally elevated activity of osteoclasts. The imbalance between osteoblasts and osteoclasts results in bone being decomposed much faster than it is synthesized, causing a significant loss of calcium from the bone tissues, reduced bone mass, and bones that are fragile and prone to fractures.

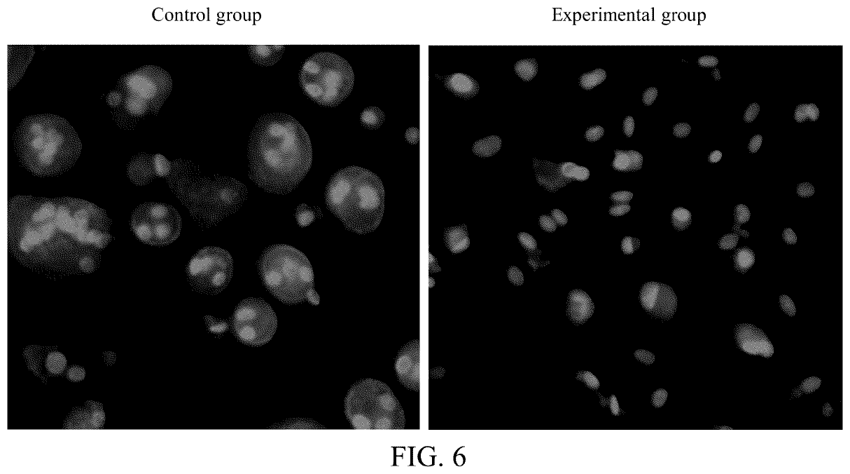

As shown in FIG. 6, compared with the control group (mature multinuclear osteoclasts), the number of osteoclasts in the experimental group (undifferentiated mononuclear cells) was significantly decreased (decreased by 70%), which indicates that the Sample 1 could significantly inhibit the differentiation and maturation of osteoclasts and effectively slow down bone loss, thereby enhancing bone density, increasing bone hardness, and promoting bone health.

### Example 8: Preparation of Mixture of Type II Collagen-Mimicking Amino Acid and Plant Extract

24.03% of glycine, 15.98% of L-proline, 7.93% of L-alanine, 5.56% of L-aspartic acid, 5.44% of L-glutamine, 4.68% of L-glutamic acid, 4.26% of L-arginine, 3.97% of L-lysine hydrochloride, 3.96% of L-serine, 3.31% of L-leucine, 2.60% of L-threonine, 2.25% of L-valine, 2.07% of L-methionine, 2.01% of L-isoleucine, 1.48% of L-phenylalanine, 0.59% of L-tyrosine, 0.47% of L-histidine, 0.41% of L-tryptophan, 3.25% of maltodextrin, 2.75% of elderberry extract (obtained by extracting fruits of Sambucus nigra, purchased from Hunan Huacheng Biotech, Inc. (Huachengbio)), and 3.00% of vitamin C were added, in weight percentage, to a mixer for mixing to obtain a primary mixture. The primary mixture was sieved by a sieve (with the pore size of 40 meshes) to obtain a fine and uniform intermediate mixed product. The intermediate mixed product was homogenized (homogenizer: BAON, G560 (2-434437)) to obtain a uniform mixed product, namely the type II collagen-mimicking amino acid and plant extract composition (Sample 2) in Example 8.

### Example 9: Preparation of Mixture of Type II Collagen-Mimicking Amino Acid and Plant Extract

24.03% of glycine, 15.98% of L-proline, 7.93% of L-alanine, 5.56% of L-aspartic acid, 5.44% of L-glutamine, 4.68% of L-glutamic acid, 4.26% of L-arginine, 3.97% of L-lysine hydrochloride, 3.96% of L-serine, 3.31% of L-leucine, 2.60% of L-threonine, 2.25% of L-valine, 2.07% of L-methionine, 2.01% of L-isoleucine, 1.48% of L-phenylalanine, 0.59% of L-tyrosine, 0.47% of L-histidine, 0.41% of L-tryptophan, 5.00% of maltodextrin, 3.00% of vitamin C, and 1.00% of Curcuma longa extract (obtained by extracting rhizomes of Curcuma longa, purchased from ARJUNA NATURALPRIVATE LTD) were added, in weight percentage, to a mixer for mixing to obtain a primary mixture. The primary mixture was sieved by a sieve (with the pore size of 40 meshes) to obtain a fine and uniform intermediate mixed product. The intermediate mixed product was homogenized (homogenizer: BAON, G560 (2-434437)) to obtain a uniform mixed product, namely the type II collagen-mimicking amino acid and plant extract composition (Sample 3) in Example 9.

### Example 10: Human Efficacy Test

In this example, the efficacy of the Sample 2 obtained in Example 8 on bone and joint health was provided.

Sample: Sample 2 in Example 8 (5 g/day).

Subjects: a total of 10 adults.

Inclusion criteria: adults at high risk of fractures (low bone density, postmenopausal women, age ≥65 years).

Method: a self control test was carried out. The subjects took 5 g of the Sample 2 before meals every day for 3 consecutive months. Bone density values were collected at month 0 and month 3. Experimental data was subjected to statistical analysis by Student's t-test in Excel (** indicates P<0.01 relative to week 0, *** indicates P<0.001 relative to week 0).

Test items: spinal bone density scan (dual-energy X-ray absorptiometry, DEXA): T-score, Z-score.

As shown in Table 3 below, the T-score reflects the comparison of bone mineral density (BMD) with the average BMD of young healthy adults of the same gender. The T-score can be used for diagnosing osteopenia and osteoporosis.

TABLE 3

| T-score | Explanation |
|---|---|
| −1 or above | Normal bone density |
| Less than −1 to greater than −2.5 | Bone loss (below normal BMD) |
| −2.5 or below | Osteoporosis (low BMD) |

As shown in Table 4 below, the Z-score compares the subject BMD with the average BMD of peers, taking into account gender and body mass index (BMI).

TABLE 4

| Z-score | Explanation |
|---|---|
| −1.5 or above | Typical BMD of peers |
| −1.5 or below | Atypical BMD, to be investigated |

### Test Results

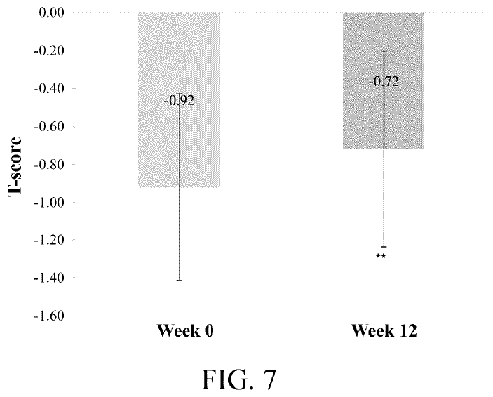

As shown in FIG. 7, after taking the Sample 2 for 12 consecutive weeks, the T-score of spinals of the subjects significantly increased from −0.92 to −0.72, with a change rate of 21.7%, and 80% of the subjects showed effective improvement.

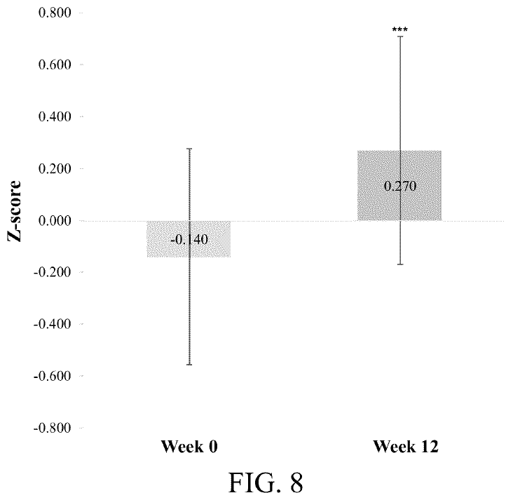

As shown in FIG. 8, after taking the Sample 2 for 12 consecutive weeks, the Z-score of spinals of the subjects significantly increased from −0.140 to 0.270, with a change rate of 293%, and 90% of the subjects showed effective improvement.

A decrease in bone mineral density is the main reason for reduced bone hardness and increased risk of fractures; and the Sample 2 can significantly enhance bone mineral density, thereby reducing the risk of fractures in menopausal women or the elderly, and it can also slow down bone loss, enhance bone strength, increase bone hardness, and improve skeletal support.

### Example 11: Human Efficacy Test

In this example, the efficacy of the Sample 2 obtained in Example 8 on bone and joint health was provided.

Sample: Sample 2 obtained in Example 8 (5 g/day)

Subjects: a total of 10 adults

Inclusion criteria: 20 years old or older.

Method: a self control test was carried out. The subjects took 5 g of the Sample 2 before meals every day for 3 consecutive months. Blood samples were collected at month 0 and month 3. Experimental data was subjected to statistical analysis by Student's t-test in Excel (* indicates P<0.05 relative to week 0).

Test item: serum TNF-α levels (inflammatory marker).

Test method: after collecting fasting blood, the samples were sent to Liren Testing Laboratory for examination. TNF-α was tested by an ELISA KIT (Elabscience, E-EL-H0109).

### Test Results

TNF-α is one of the key inflammatory factors in occurrence and development of arthritis. TNF-α can not only directly damage chondrocytes but also stimulate synovial fibroblasts and chondrocytes to produce inflammatory mediators including matrix metalloproteinases (MMPs), accelerate degradation of extracellular matrix, and lead to joint cartilage destruction and persistent inflammation.

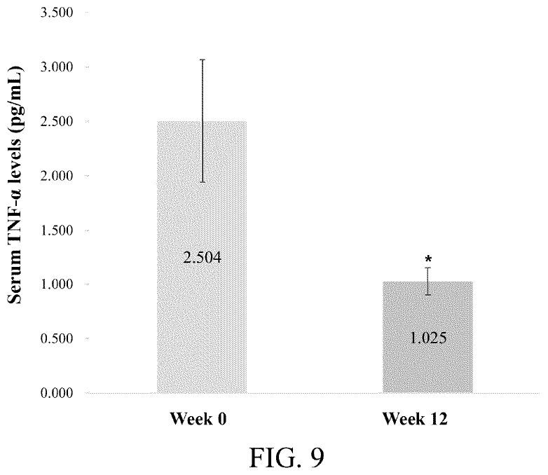

As shown in FIG. 9, after taking the Sample 2 for 12 consecutive weeks, TNF-α levels decreased by 59.1%, and 90% of the subjects showed effective improvement. Therefore, the Sample 2 can reduce the levels of inflammatory factors, thereby helping to prevent inflammatory responses.

### Example 12: Human Efficacy Test

In this example, the efficacy of the Sample 3 obtained in Example 9 on bone and joint health was provided.

Sample: Sample 3 obtained in Example 9 (5 g/day)

Subjects: a total of 10 adults Inclusion criteria: Adults aged 20 or older with joint discomfort.

Method: a self control test was carried out. The subjects took 5 g of the sample before meals every day for 3 consecutive months. Self-assessment questionnaires were collected at week 0, week 4, and week 12. Measurement method: in the self-assessment questionnaire, a WOMAC (The Western Ontario and McMaster Universities Osteoarthritis Index) total score, a joint pain score, a stiffness score, and an activity score were surveyed. Experimental data was subjected to statistical analysis by Student's t-test in Excel (* indicates P<0.05 relative to week 0, ** indicates P<0.01 relative to week 0).

### Test Results

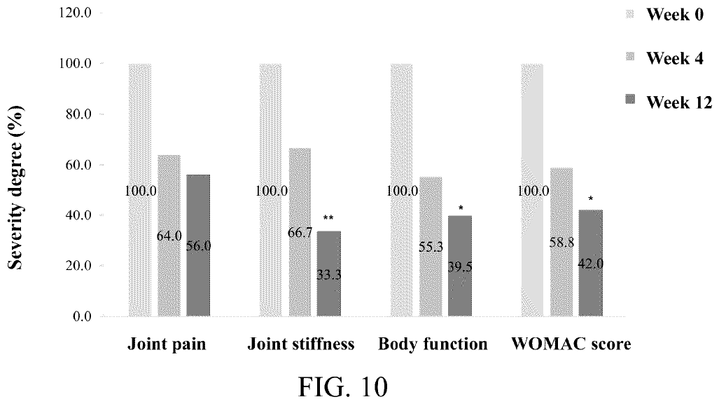

As shown in FIG. 10, after taking the Sample 3 for 12 consecutive weeks, the WOMAC score decreased significantly by 58%, the joint pain score decreased by 44%, the joint stiffness score decreased by 66.7%, and the activity score (physical function) decreased by 60.5%, which indicates that the composition of Sample 3 can effectively relieve joint stiffness in people with joint discomfort, thereby improving joint mobility, and can effectively relieve joint pain to improve joint function and physical performance.

What is claimed is:

1. A method for increasing bone hardness, comprising: administering an effective dose of a mixture to a subject in need thereof, wherein the mixture comprises an amino acid composition and a berry extract, and the amino acid composition comprises the following amino acids or salts thereof: 20.0%-28.0% of glycine, 13.0%-18.0% of proline, 6.0%-10.0% of alanine, 4.0%-7.0% of aspartic acid, 4.0%-7.0% of glutamine, 3.5%-6.0% of glutamic acid, 3.5%-5.0% of arginine, 3.0%-4.5% of lysine, 3.0%-4.5% of serine, 2.5%-4.0% of leucine, 2.0%-3.2% of threonine, 1.8%-2.8% of valine, 1.5%-2.5% of methionine, 1.5%-2.5% of isoleucine, 1.0%-2.0% of phenylalanine, 0.4%-0.8% of tyrosine, 0.3%-0.6% of histidine, 0.3%-0.6% of tryptophan, and an optional additive.

2. The method according to claim 1, wherein the mixture is used for enhancing bone mineral density.

3. The method according to claim 1, wherein the mixture is used for promoting osteoblast differentiation.

4. The method according to claim 1, wherein the mixture is used for inhibiting osteoclast differentiation.

5. The method according to claim 1, wherein based on the total weight of the mixture being 100%, the berry extract accounts for 2.0%-3.5% in weight percentage.

6. The method according to claim 1, wherein the berry extract is an elderberry extract.

7. A method for preventing inflammatory responses, comprising: administering an effective dose of a mixture to a subject in need thereof, wherein the mixture comprises an amino acid composition and a berry extract, and the amino acid composition comprises the following amino acids or salts thereof: 20.0%-28.0% of glycine, 13.0%-18.0% of proline, 6.0%-10.0% of alanine, 4.0%-7.0% of aspartic acid, 4.0%-7.0% of glutamine, 3.5%-6.0% of glutamic acid, 3.5%-5.0% of arginine, 3.0%-4.5% of lysine, 3.0%-4.5% of serine, 2.5%-4.0% of leucine, 2.0%-3.2% of threonine, 1.8%-2.8% of valine, 1.5%-2.5% of methionine, 1.5%-2.5% of isoleucine, 1.0%-2.0% of phenylalanine, 0.4%-0.8% of tyrosine, 0.3%-0.6% of histidine, 0.3%-0.6% of tryptophan, and an optional additive.

8. The method according to claim 7, wherein the mixture is used for reducing a level of inflammatory factors.

9. The method according to claim 7, wherein the mixture is used for preventing chondrocyte inflammation.

10. The method according to claim 7, wherein based on the total weight of the mixture being 100%, the berry extract accounts for 2.0%-3.5% in weight percentage.

11. The method according to claim 7, wherein the berry extract is an elderberry extract.

12. A method for relieving joint pain, comprising: administering an effective dose of a mixture to a subject in need thereof, wherein the mixture comprises an amino acid composition and a Curcuma plant extract, and the amino acid composition comprises the following amino acids or salts thereof: 20.0%-28.0% of glycine, 13.0%-18.0% of proline, 6.0%-10.0% of alanine, 4.0%-7.0% of aspartic acid, 4.0%-7.0% of glutamine, 3.5%-6.0% of glutamic acid, 3.5%-5.0% of arginine, 3.0%-4.5% of lysine, 3.0%-4.5% of serine, 2.5%-4.0% of leucine, 2.0%-3.2% of threonine, 1.8%-2.8% of valine, 1.5%-2.5% of methionine, 1.5%-2.5% of isoleucine, 1.0%-2.0% of phenylalanine, 0.4%-0.8% of tyrosine, 0.3%-0.6% of histidine, 0.3%-0.6% of tryptophan, and an optional additive.

13. The method according to claim 12, wherein the mixture is used for preventing joint wear.

14. The method according to claim 13, wherein the mixture is used for promoting secretion of cartilage collagen.

15. The method according to claim 13, wherein the mixture is used for promoting secretion of glucosaminoglycans.

16. The method according to claim 12, wherein based on the total weight of the mixture being 100%, the Curcuma plant extract accounts for 0.5%-3.0% in weight percentage.

17. The method according to claim 12, wherein the berry extract is an elderberry extract.

18. A method for improving joint mobility, comprising: administering an effective dose of a mixture to a subject in need thereof, wherein the mixture comprises an amino acid composition and a Curcuma plant extract, and the amino acid composition comprises the following amino acids or salts thereof: 20.0%-28.0% of glycine, 13.0%-18.0% of proline, 6.0%-10.0% of alanine, 4.0%-7.0% of aspartic acid, 4.0%-7.0% of glutamine, 3.5%-6.0% of glutamic acid, 3.5%-5.0% of arginine, 3.0%-4.5% of lysine, 3.0%-4.5% of serine, 2.5%-4.0% of leucine, 2.0%-3.2% of threonine, 1.8%-2.8% of valine, 1.5%-2.5% of methionine, 1.5%-2.5% of isoleucine, 1.0%-2.0% of phenylalanine, 0.4%-0.8% of tyrosine, 0.3%-0.6% of histidine, 0.3%-0.6% of tryptophan, and an optional additive.

19. The method according to claim 18, wherein the mixture is used for relieving joint stiffness.

20. The method according to claim 18, wherein the mixture is used for promoting secretion of cartilage collagen.

21. The method according to claim 18, wherein the mixture is used for promoting secretion of glucosaminoglycans.

22. The method according to claim 18, wherein based on the total weight of the mixture being 100%, the Curcuma plant extract accounts for 0.5%-3.0% in weight percentage.

23. The method according to claim 18, wherein the Curcuma plant extract is a Curcuma longa extract.


SEQUENCE LISTING (14 sequences)

SEQ ID NO: 1 - DNA, 22 bp, synthetic construct
  source 1..22: mol_type = other DNA
gctggtgaagaaggcaaacgag

SEQ ID NO: 2 - DNA, 22 bp, synthetic construct
  source 1..22: mol_type = other DNA
ccatcttgacctgggaatccac

SEQ ID NO: 3 - DNA, 20 bp, synthetic construct
  source 1..20: mol_type = other DNA
gggatacagagcagagaccc

SEQ ID NO: 4 - DNA, 20 bp, synthetic construct
  source 1..20: mol_type = other DNA
ggggtacagcagagagatag

SEQ ID NO: 5 - DNA, 20 bp, synthetic construct
  source 1..20: mol_type = other DNA
ttcgacttctctccagccga

SEQ ID NO: 6 - DNA, 20 bp, synthetic construct
  source 1..20: mol_type = other DNA
tccactggcctgatccatgt

SEQ ID NO: 7 - DNA, 18 bp, synthetic construct
  source 1..18: mol_type = other DNA
ttgctgctgtacctttcg

SEQ ID NO: 8 - DNA, 20 bp, synthetic construct
  source 1..20: mol_type = other DNA
tgcttctgctgatctcactt

SEQ ID NO: 9 - DNA, 20 bp, synthetic construct
  source 1..20: mol_type = other DNA
ttgggcctcaaaggaaagaa

SEQ ID NO: 10 - DNA, 23 bp, synthetic construct
  source 1..23: mol_type = other DNA
tgtgaggtgctgatgtaccagtt

SEQ ID NO: 11 - DNA, 17 bp, synthetic construct
  source 1..17: mol_type = other DNA
cccaaggcgccacatct

SEQ ID NO: 12 - DNA, 23 bp, synthetic construct
  source 1..23: mol_type = other DNA
caccccgaagttcagtagacaga

SEQ ID NO: 13 - DNA, 19 bp, synthetic construct
  source 1..19: mol_type = other DNA
cctcagttctgcgcctttg

SEQ ID NO: 14 - DNA, 19 bp, synthetic construct
  source 1..19: mol_type = other DNA
tgaagcgtttcgggatctg